(19) United States
(12) Patent Application Publication
(10) Pub. No.: US 2014/0303426 A1
(43) Pub. Date: Oct. 9, 2014

(54) IMPLANTABLE BLOOD PUMP, BLOOD PUMP SYSTEM, AND METHOD FOR DATA TRANSFER IN A BLOOD PUMP SYSTEM

(71) Applicant: CIRCULITE, INC., Teaneck, NJ (US)

(72) Inventors: Wolfgang Kerkhoffs, Aachen (DE); Oliver Marseille, Aachen (DE); J. Christopher Flaherty, Auburndale, FL (US)

(73) Assignee: CIRCULITE, INC., Teaneck, NJ (US)

(21) Appl. No.: 14/242,090

(22) Filed: Apr. 1, 2014

Related U.S. Application Data
(60) Provisional application No. 61/867,686, filed on Aug. 20, 2013.

(30) Foreign Application Priority Data
Apr. 5, 2013 (DE) 102013005767.1
Aug. 20, 2013 (DE) 102013013700.4

(51) Int. Cl. A61M 1/10; A61M 1/12

(52) U.S. Cl. CPC A61M 1/1086; A61M 1/122
USPC 600/16

(57) ABSTRACT

An implantable blood pump, a blood pump system, and a method for data transfer in a blood pump system. By integrating the electronic components into the blood pump, they are located in the direct vicinity of corresponding component parts. An accumulation of heat is prevented by arranging the electronics on blood-guiding components of the blood pump, in particular on the cannula. This results in efficient cooling of the electronic components.

### CROSS-REFERENCE TO RELATED APPLICATION

This application claims the priority of German Application No. 10 2013 005 767.1 filed Apr. 5, 2013 (pending) and German Application No. 10 2013 013 700.4 filed Aug. 20, 2013 (pending). This application also claims the benefit of U.S. Provisional Application Ser. No. 61/867,686 filed Aug. 20, 2013 (pending). The disclosures of each of these priority applications are hereby incorporated by reference herein.

### TECHNICAL FIELD

The invention relates to an implantable blood pump, a blood pump system, and a method for data transfer in a blood pump system.

### BACKGROUND

Implantable blood pump systems are known from the prior art. In these systems, the electronics, in particular the power electronics but also sensor electronics, which may be provided, are generally arranged outside the body since they may lead to a development of heat during operation, which has to be removed by cooling. Only the coils of the actuator motors or the electromagnets, sensors and supply lines are normally also implanted. In the case of published concepts of fully implantable systems with transcutaneous energy supply, the electronics are always accommodated in separate modules in their own housing. These housings give off the heat to the surrounding tissue.

### SUMMARY

The object of the present invention is to improve such blood pumps from the prior art.

This is achieved in accordance with a first aspect of the invention by an implantable blood pump, wherein electronics are integrated into the blood pump. This has the advantage that the electronics are located in the direct vicinity of the corresponding component parts. In particular, the electronics may consist of component parts that switch the electrical currents, wherein heat may develop. Normally only the electronic components that necessarily have to be integrated with the actuator (motor or electromagnet) or sensor system on the pump are placed in the vicinity of the blood-conveying channels. Electronics in the meaning of the invention above all describe the components that are usually arranged on the outside of the body (e.g., switching transistors, MOSFETs, power controllers, voltage controllers). In accordance with the invention, the term electronics expressly does not describe direct actuators (motors, moving coil drives etc.) or transmission systems for mechanical actions (gears, lever etc.).

The electronics are advantageously arranged on, or in the vicinity of, blood-conveying components of the blood pump or of the overall system. In particular, these components of the blood pump should be heat-conductive. Between the components and the bloodstream there are preferably only two direct material transitions, particularly preferably only one direct metal transition, while preventing air or gases acting as thermal insulation. Connection by way of heat-conducting adhesives and or casting compound is advantageous. There may be a suitable material selection or shaping, for example in the form of a heat exchanger. It is also of advantage to place the electronic components on different or opposite blood-conveying walls or to connect them in a heat-conducting manner. The heat created during use of the electronics can thus be dissipated into the blood. This effectively cools the integrated electronics as a result. The distance between the blood-conveying walls can be smaller, or even considerably smaller than the diagonal of the surface area of the electronic components facing the blood. More particularly, the distance between the components and the blood-conveying wall can be smaller than the diameter of the components. A transfer of the heat from the electronic components to the blood-conveying walls through direct heat conduction and not through free or forced (pumped) convection via fluids or gases is advantageous.

The electronics may be a motor controller and/or a power controller and/or a sensor controller, in particular for a flow sensor system and/or a temperature sensor system and/or a pressure sensor system. In this case, the motor controller is understood to mean the controller for the motor electronics, the sensor controller is understood to mean the controller for the sensor electronics and the power controller is understood to mean the controller for the power supply of the electronics as a whole. These could be integrated into the blood pump individually or in combination. The motor controller and the power controller can be summarized by the term “power electronics” and are arranged in a bundle accordingly. In these components the heat can be generated through constant or cyclical ohmic losses, as well as through chemical/galvanic or piezo-electrical conversions. Internal friction within the components is also conceivable as a heat generator.

The blood pump advantageously has an implantable power supply. This has the advantage that it can be operated independently of an external power supply, at least temporarily.

A second, likewise independent, inventive aspect of the invention concerns a blood pump system, in particular with an implantable blood pump and an external controller. This extracorporeal controller is often equipped with complex algorithms and software. The extracorporeal arrangement is therefore advantageous, since software updates and/or extensions, or additions to the algorithms can be implemented externally without risk.

The electronics are advantageously connected to the external controller via an RF link or other wireless transfer device. Wireless transfer of data from the electronics to the controller is thus possible.

It is advantageous if the external unit has an alarm device. If the external controller is distanced from the patient, an alarm can thus be emitted. An alarm that is triggered by the implanted electronics can also be emitted by the external alarm device.

It is also advantageous if the blood pump has an emergency mode function. This ensures that the blood pump functions even if the external controller is omitted.

A further, likewise independent, inventive aspect of the invention concerns a blood pump system, wherein the blood pump and the external controller are connected via a transcutaneous cable or a TET (transcutaneous energy transfer) interface and the data of the sensor controller are modulated onto the motor current. In particular, the cable may be a three-pole transcutaneous cable. Corresponding modulation, by means of a signal converter, of the data of the sensor controller onto the contact of the cable guiding the motor current has the advantage that it is not necessary to provide a separate contact for this signal. This leads to a reduction in the number of cables at the opening in the skin. Likewise, the number of poles at the plug connector can be reduced. Alternatively, in the case of a TET interface also, the data of the sensor controller may likewise be modulated by means of a signal converter onto the line guiding the motor current.

In accordance with a further, likewise independent, inventive aspect of the invention, a method for data transfer in a blood pump system, wherein the data of the sensor controller are modulated onto the motor current, is advantageous.

As an additional, independent inventive aspect, the electronics include a heat generating element positioned proximate a blood flow pathway and at least one sensor for measuring temperature. Heat generated by the heat generating element and/or blood flow rate through the blood pump is modified in response to a temperature sensed by the sensor.

As an additional, independent inventive aspect, the electronics regulate heat generation based on a temperature reading taken in or proximate to the blood pump. As one example, the electronics may regulate heat generation continuously, such as in a closed loop manner, and/or heat generation is regulated in accordance with blood flow rate. At least one threshold temperature may be established in the electronics and the electronics are down-regulated if the threshold temperature is surpassed and/or blood flow rate through the pump is increased if the threshold temperature is surpassed. Optionally, at least a first threshold temperature and a second, higher threshold temperature are established in the electronics, and a first amount of down-regulation and/or blood flow rate increase is provided if the first threshold temperature is surpassed, and a second, larger amount of down-regulation and/or blood flow rate increase is provided if the second threshold temperature is surpassed.

As an additional, independent inventive aspect, the electronics further comprise multiple electronic subassemblies distributed to avoid relatively higher heat generating subassemblies being next to each other.

As an additional, independent inventive aspect, fins are thermally coupled to the electronics.

As an additional, independent inventive aspect, the pump further comprises a pump housing and the electronics further comprise a module at least partially positioned about the pump housing. A major axis of the module is oriented relatively orthogonal to blood flow through the housing to thereby minimize the amount of heat received by each blood cell passing through the housing. The housing may include a thinned wall portion at heat transfer locations and/or the housing may include a minimum wall thickness at heat transfer locations for preventing undesired temperature spikes.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

FIG. 1 shows a blood pump system with electronics integrated into the blood pump and an external controller, connected via a transcutaneous cable.

FIG. 2 shows a blood pump system with electronics integrated into the blood pump and an external controller, connected via an RF link.

FIG. 3 shoes a blood pump system with integrated electronics and an external controller, connected via an RF link to an integrated power supply.

FIG. 4 shows a blood pump system with an additional power controller integrated into the pump.

FIG. 5 shows a blood pump system, in which the electronic data are transferred by modulation onto the current signal with the presence of a TET interface.

FIG. 6 shows a blood pump system, in which the electronic data are transferred via an RF link.

FIG. 7 shows a blood pump system with an integrated power controller, sensor controller and motor controller, in which the electronic data are transferred by modulation via the TET interface.

FIG. 8 shows a blood pump system in which the electronic data are transferred via an RF link.

FIG. 9 shows a device including a pumping assembly in accordance with further aspects or illustrative embodiments of the invention.

FIG. 10 shows a device including a pumping assembly in accordance with further aspects or illustrative embodiments of the invention.

FIG. 11 shows a device including a pumping assembly in accordance with further aspects or illustrative embodiments of the invention.

### DETAILED DESCRIPTION

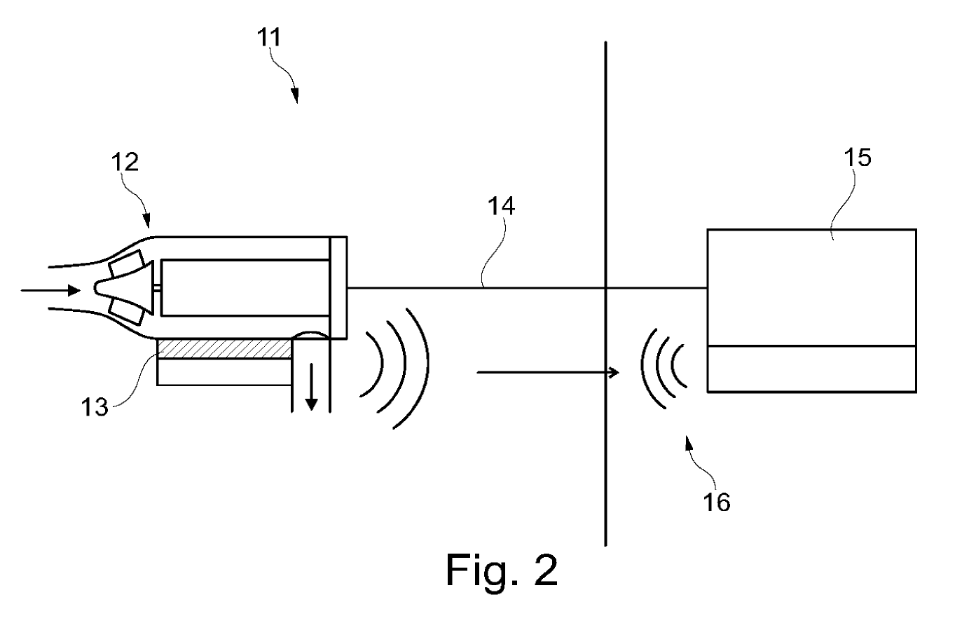

Both in the blood pump system 1 and in the blood pump system 11 (FIG. 2), the blood pump 2, 12 has an integrated sensor controller 3, 13. This is arranged at the blood pump 2, 12 in the vicinity of the blood flow on the pump housing so that heat can be removed effectively. The blood pump 2 is connected in each case to a controller 5, 15 by means of a transcutaneous cable 4, 14 via a connector 6 (not shown in FIG. 2). In this case, the sensor data captured by the sensor controller 3, 13, such as flow data, temperature data or pressure data for example, can be transferred via an RF link (16, see FIG. 2) or by modulation onto the motor connection, or via another type of wireless communication link. For this purpose, the signal is converted by a signal converter 7 or 8 respectively, such as when signal converter 7 comprises a modulator and signal converter 8 comprises a de-modulator.

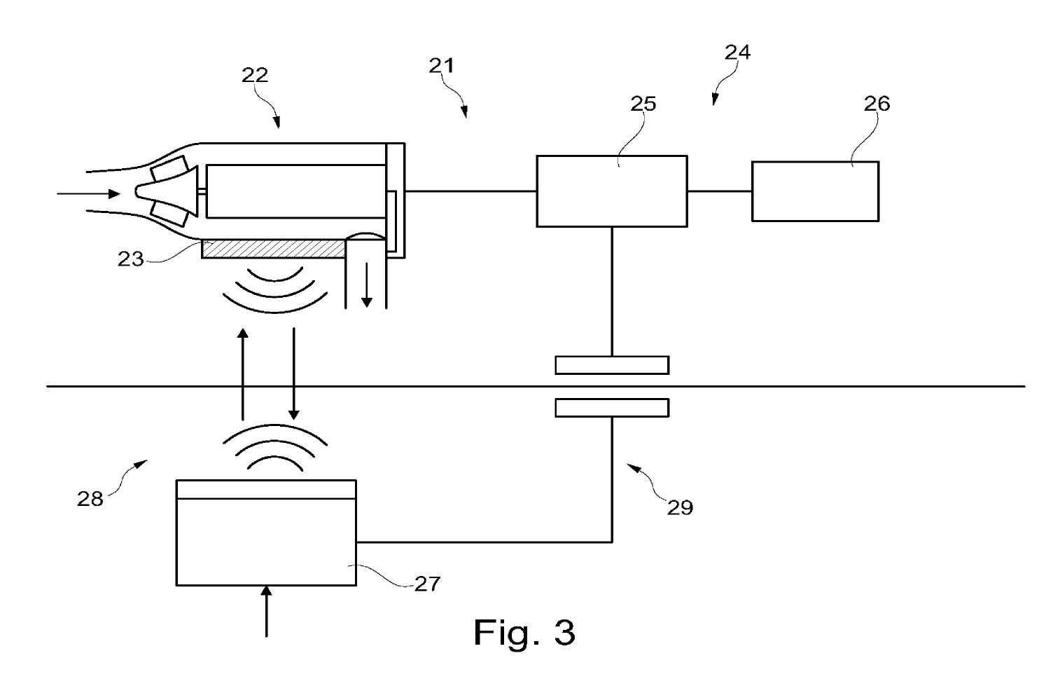

The motor controller 23 may also be integrated into the blood pump 22, as in the blood pump system 21 (see FIG. 3). In addition, the blood pump 22 in this blood pump system 21 has an implantable power supply 24, with a power management system 25 and a battery 26. Similarly to the blood pump 22, these are likewise implantable. Communication with the external controller 27 is achieved via an RF link 28. With constantly applied voltage, the current changes according to load. In another aspect, current changes according to load even if voltage is varied. In this regard, motor information may be decoded as long as applied voltage is known. Information concerning the state of the pump can thus be obtained. The power supply is achieved via a TET connection 29, such as a wireless transfer of energy selected from the group consisting of: electromagnetic energy such as electromagnetic energy delivered by an external coil and received by an implanted coil; light energy such as light energy delivered by an external light emitting element and received by an implanted light receiving element; sound energy such as sound energy delivered by an external sound transducer and received by an implanted sound transducer; and combinations of these.

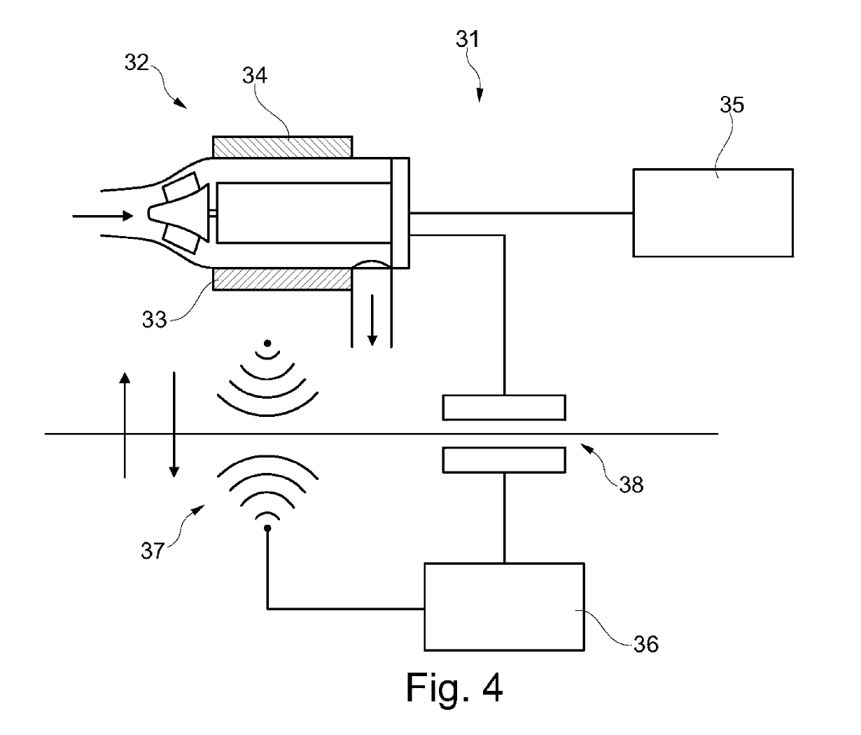

Referring to the system 31 of FIG. 4, in addition to the motor controller 33, the power controller 34 may also be integrated into the blood pump 32. In this case too, an implantable battery 35 can be provided. The connection to the external controller 36 is also achieved in this instance via an RF link 37 and a TET connection 38.

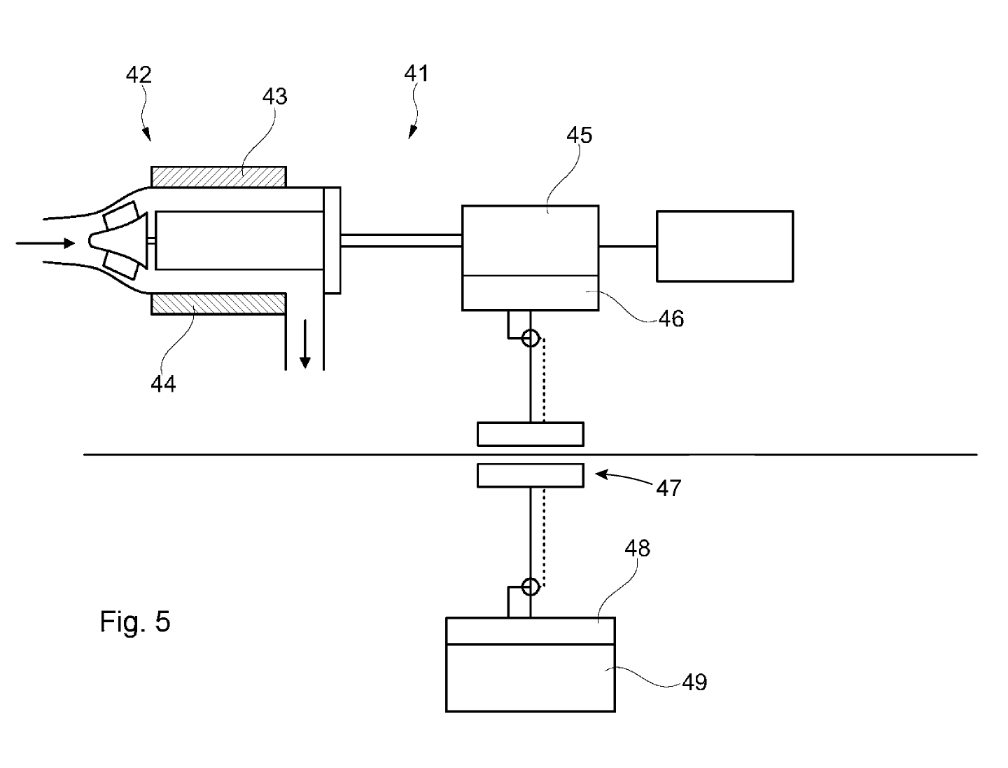

To further simplify the blood pump system 41, the electronic data from the sensor controller 43 and motor controller 44, which are integrated into the blood pump 42, can be modulated onto the TET connection 47 acting as a power supply via a power manager 45 and a signal converter 46 and can then be made available to the external controller 49 via a signal converter 48, as shown in FIG. 5.

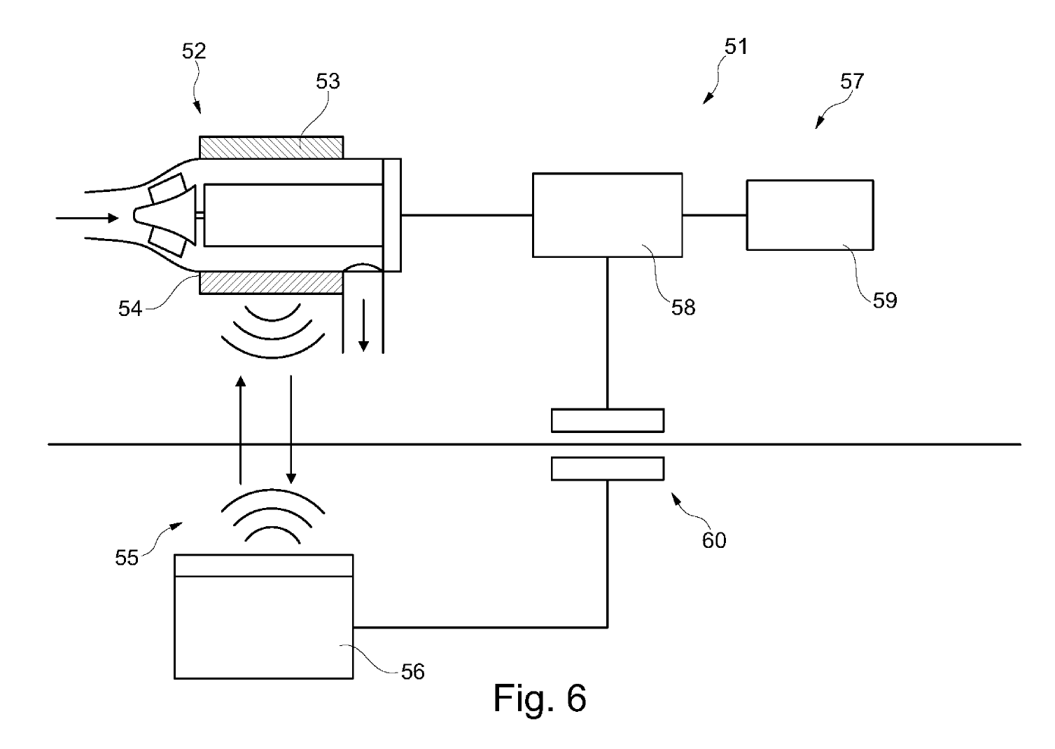

Alternatively however, it is of course also possible to transfer the data from the sensor controller 53 and motor controller 54, which are both integrated into the blood pump 52, to an external controller 56 via an RF link 55, as illustrated in the blood pump system 51 in FIG. 6. Said external controller is connected via a TET connection 60 to the power supply 57, which is likewise implanted and consists of a power manager 58 and battery 59.

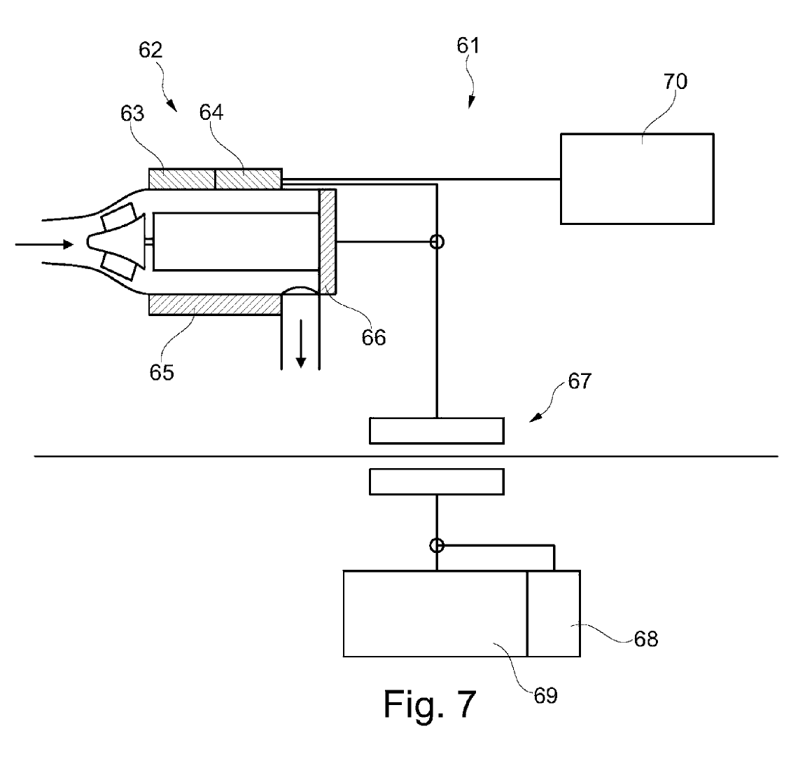

Lastly, all electronic components, such as the power controller 63, sensor controller 64 and motor controller 65, can be integrated into the blood pump 62 of system 61, as shown in FIG. 7, such as to effectively remove heat from at least one of power controller 63, sensor controller 64 and/or motor controller 65 as has been described above. The corresponding electronic data are modulated onto the TET connection 67 via the signal converter 66 and are fed into the external controller 69 via the signal converter 68. In this case too, the battery 70 can be implantable.

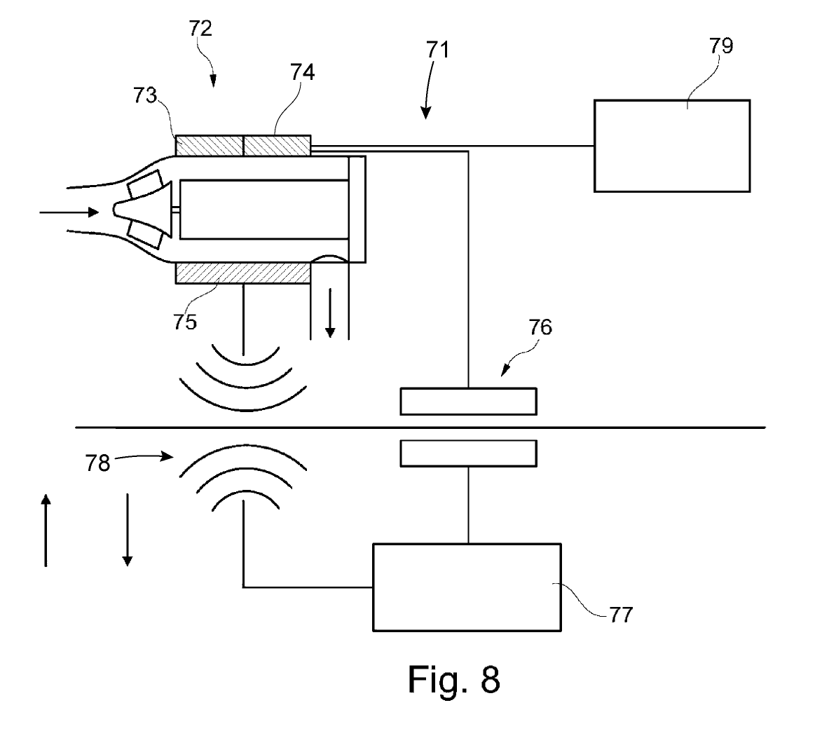

Alternatively, it is possible to design a blood pump system 71 such that the data from the power controller 73, sensor controller 74 and/or motor controller 75 integrated into the blood pump 72 are not to be transferred to the external controller 77 via the TET connection 76, but an additional RF link 78 is to be used for this purpose, as can be seen in FIG. 8. In this case too, the battery 79 can be implantable.

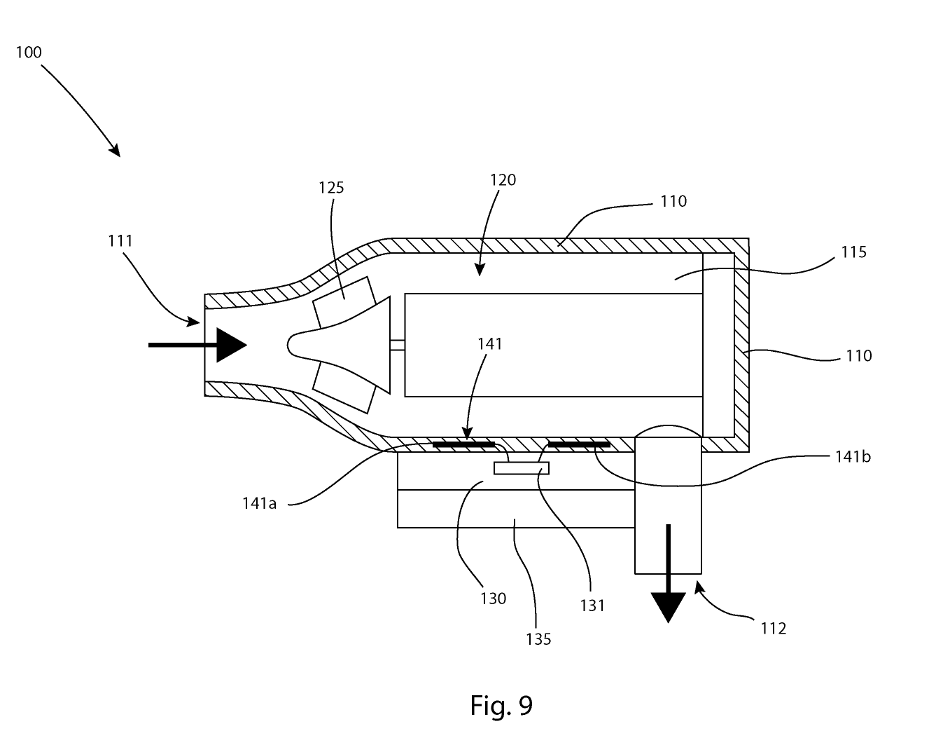

As shown in FIG. 9, device 100 includes an implantable housing 110. Housing 110 is a biocompatible material configured for implantation, such as a biocompatible metal (e.g., stainless steel or titanium) and/or a biocompatible plastic (e.g., polysulfone or polyoxymethylene). Housing 110 includes an inlet 111 and an outlet 112. Inlet 111 and outlet 112 are configured to each attach to a flexible conduit (e.g., a flexible cannula or other tube) for attachment to one or more blood locations, such as the left atrium and an artery, respectively. Housing 110 surrounds a pumping assembly 120 which includes rotor 125. A chamber 115 is located between housing 110 and pumping assembly 120, such that rotation of rotor 125 causes blood to flow from inlet 111 to outlet 112.

Device 100 further includes an electronics module 130 comprising electronic componentry and circuitry such as: pump assembly control circuitry; switching, rectification, and/or other power conversion circuitry; voltage control, current control and/or other power regulation circuitry; telemetry circuitry such as RF; optical such as infrared, magnetic sound such as ultrasound or other wireless communication means; microcontroller and/or microprocessor circuitry; memory circuitry; and/or sensor interface circuitry, such as sensor interface circuitry 131. In some embodiments, device 100 comprises an implantable power supply, battery 135, which can provide power to electronics module 130 and/or pumping assembly. Alternatively or additionally, an external power supply can be included.

Device 100 can include one or more sensors, such as sensors 141a and 141b (collectively sensor 141) shown positioned on or within housing 110 proximate electronics module 130 and chamber 115. Sensors 141a and 141b can be similar or dissimilar sensors. Sensors 141a and 141b (collectively sensor 141) are operably connected to sensor interface circuitry 131 of electronics module 130, such as via one or more wires. In some embodiments, sensors 141 can comprise a temperature sensor, such as a thermocouple or a thermister configured to measure the temperature of housing 110 at a location proximate electronics module 130. Alternatively or additionally, sensors 141 can comprise a sensor selected from the group consisting of: a flow sensor; a pressure sensor; a magnetic sensor such as a hall effect sensor; and combinations thereof. Sensors 141a and 141b can be operably attached to sensor interface circuitry 131.

Electronics module 130 is attached to or otherwise positioned proximate housing 110, such that heat generated by electronics module 130 and/or battery 135, and is absorbed by blood passing through chamber 115. In some embodiments, electronics module 130 and/or battery 135 can be constructed and arranged such that the heat generated by all or a portion of either can be regulated (e.g., by drawing less current or otherwise modifying function). In these embodiments, the heat generated can be regulated based on one or more temperature measurements determined by electronics module 130 via signals obtained from sensor 141. In some embodiments, heat generation is continually regulated by electronics module 130, such as in a closed loop fashion based on one or more temperature measurements received from sensor 141. For example, at higher flow rates, more heat can be generated due to an increase in electronic functions (e.g., a higher sampling rate is used) and/or more electronic functions can be performed, due to the increased cooling effect of the increased blood flow rate through chamber 115. Conversely, at lower flow rates, electronic module 130 can down-regulate one or more functions to produce less heat. Alternatively or additionally, electronics module 130 can be configured such that when a measured temperature reaches a pre-determined threshold, one or more heat-generating functions of electronics module 130 may be stopped or reduced, such as to reduce heat production. In some embodiments, a first temperature threshold causes a decrease in one or more heat-generating functions, and a second (e.g., higher) temperature threshold causes a stoppage of one or all heat-generating functions. The temperature threshold may be set to prevent damage to blood, such as a temperature threshold configured to prevent blood from being heated above 38° C., 39° C., 42° C. or 45° C. Alternatively or additionally, when a measured temperature exceeds a threshold, the blood flow rate generated by pumping assembly 120 may be increased, such as to improve the absorption of heat by blood passing through chamber 115.

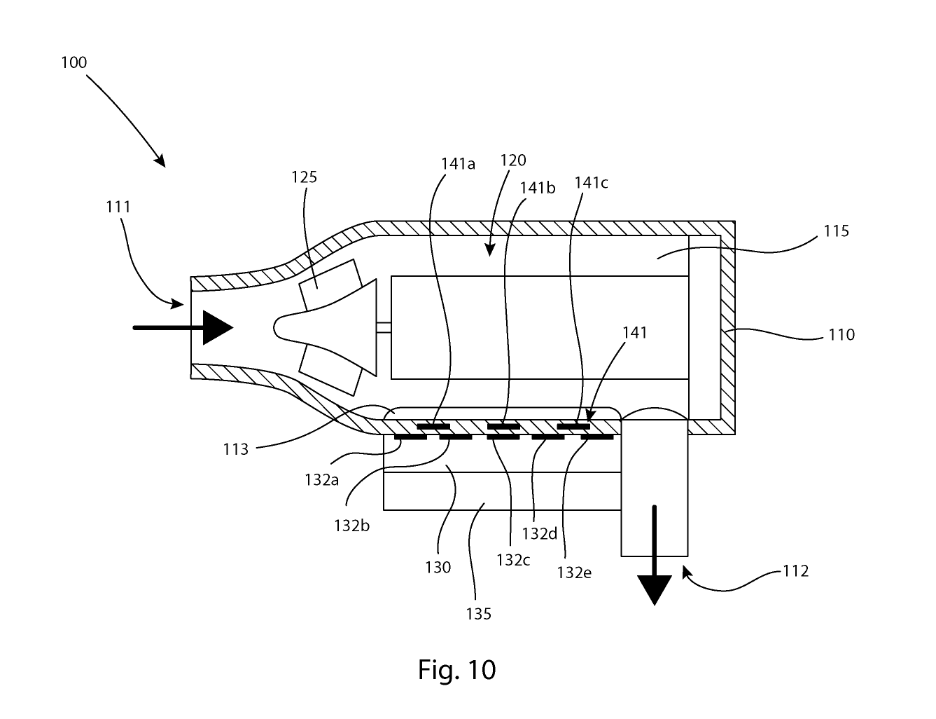

Device 100 of FIG. 10 can be of similar construction and arrangement as Device 100 of FIG. 9. In FIG. 10, electronics module 130 includes sub-assemblies 132a, b, c, d and e. Sub-assemblies 132a, 132c and 132e under standard and/or non-standard conditions (e.g., normal operation or under an alarm condition) produce more heat than sub-assemblies 132b and 132d. The alternating positions are used to avoid excessive heat being produced in one location, such as to avoid damaging blood passing through chamber 115.

In some embodiments, and as also shown in FIG. 10, device 100 comprises one or more fins 113 thermally attached to housing 110, electronics module 130 and/or battery 135 such as to cause the spread of heat (e.g., to avoid a “hot spot” and/or otherwise spread heat out over a larger surface area) and/or to increase the rate of absorption of the heat, such as when fin 113 is positioned in the flow of blood as shown in FIG. 10. Fins 113 can include smooth edges and/or contours to avoid creating turbulence or otherwise damaging blood passing thereby.

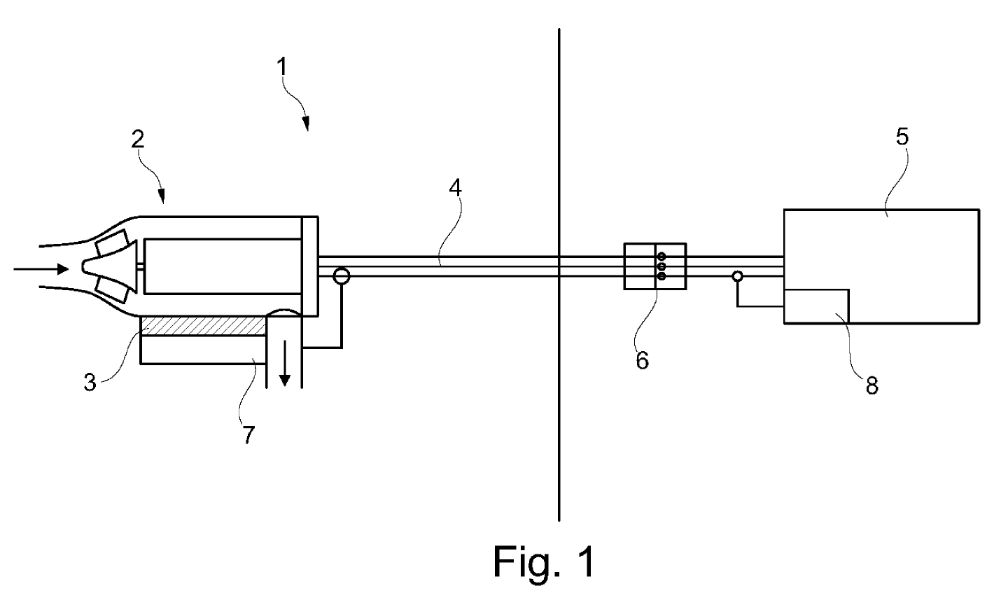

Device 100 can comprise one or more sensors, such as sensors 141a-c shown. Sensors 141a-c can comprise a temperature sensor or other sensor and can be operably connected to electronics module 130, such as via one or more wires not shown. Electronics module 130 can regulate heat output based on one or more temperature readings, such as has been described above in reference to FIG. 1.

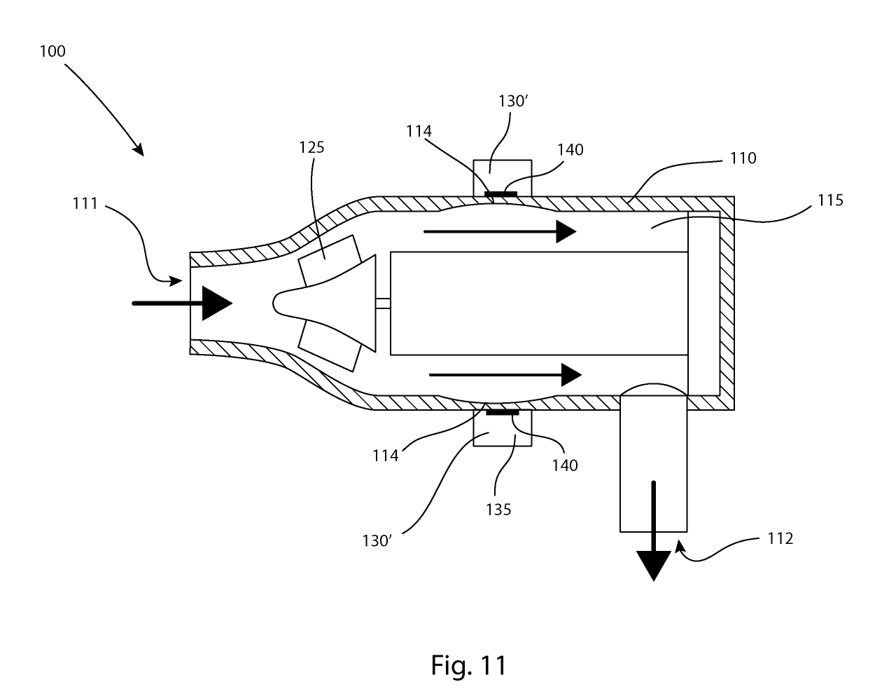

Device 100 of FIG. 11 can be of similar construction and arrangement as Device 100 of FIG. 9 and/or FIG. 10. In FIG. 11, electronics module 130′ comprises a circumferential assembly (or partial circumferential assembly, such as an assembly traversing between 90° and 360° of the circumference of housing 110). The major axis of electronics module 130′ is positioned relatively orthogonal to the blood flow pathway. In this relatively orthogonal direction, blood passing by the portion of housing 110 attached to electronics module 130′ has limited exposure to heat (e.g., flow path in which heat is absorbed is short).

Housing 110 can include one or more modified portions proximate electronics module 130′ and/or battery 135, such as to modify the transfer of heat into blood passing thereby. The modified portion can comprise a portion with different materials or geometry of construction, such as a reduced thickness portion, such as housing portion 114 shown positioned at the attachment location of electronics module 130′. The reduced thickness of portion 114 provides increased transfer of heat from electronics module 130′ into the passing blood, such as to reduce the temperature of housing 110 at locations proximate portion 114 (e.g., to avoid a longer pathway of heat transfer along housing 110). In some embodiments, portion 114 and/or another portion of housing 110 proximate electronics module 130′ and/or battery 135 has a minimum thickness, such as to avoid any rapid undesired heat transfer that might damage the blood passing thereby.

Device 100 can comprise a sensor array 140, such as an array of temperature sensors positioned about the circumference of housing 110 as shown, and connected to electronics module 130′ via one or more wires, not shown. Electronics module 130′ receives one or more signals from assembly 140 correlating to a circumferential distribution of temperatures about housing 110. Sensor array 140 may comprise one or more additional (e.g., non-temperature sensors), such as are described above. Electronics module 130′ can regulate heat output such based on one or more temperature readings, such as has been described above in reference to FIG. 1.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features of the invention may be used alone or in any combination depending on the needs and preferences of the user. This has been a description of the present invention, along with the preferred methods of practicing the present invention as currently known. However, the invention itself should only be defined by the appended claims.

What is claimed is:

1. An implantable blood pump, characterized in that electronics are integrated into the blood pump.

2. The blood pump according to claim 1, characterized in that the electronics are arranged on, or in the vicinity of, blood-conveying components of the blood pump.

3. The blood pump according to claim 1, characterized in that the electronics are a motor controller and/or a power controller and/or a sensor controller, in particular for a flow sensor system and/or a temperature sensor system and/or a pressure sensor system.

4. The blood pump according to claim 1, further comprising an implantable power supply coupled to the blood pump.

5. A blood pump system comprising an implantable blood pump according to claim 1 coupled with an external controller.

6. The blood pump system according to claim 5, characterized in that the electronics are connected to the external controller via a wireless link.

7. The blood pump system according to claim 5, further comprising an alarm device coupled with the external controller.

8. The blood pump system according to claim 5, wherein the external controller has an emergency mode function.

9. The blood pump system according to claim 5, wherein the blood pump and the external controller are connected via a transcutaneous cable or a TET interface, the electronics further comprises a sensor controller and the data of the sensor controller are modulated onto the motor current.

10. The blood pump according to claim 1, wherein the electronics include a heat generating element positioned proximate a blood flow pathway and at least one sensor for measuring temperature, and wherein at least one of heat generated by the heat generating element or blood flow rate through the blood pump is modified in response to a temperature sensed by the sensor.

11. The blood pump according to claim 1, wherein the electronics regulate heat generation based on a temperature reading taken in or proximate to the blood pump.

12. The blood pump according to claim 11, wherein the electronics regulate heat generation continuously, such as in a closed loop manner, and/or heat generation is regulated in accordance with blood flow rate.

13. The blood pump according to claim 11, wherein at least one threshold temperature is established in the electronics and the electronics are down-regulated if the threshold temperature is surpassed and/or blood flow rate through the pump is increased if the threshold temperature is surpassed.

14. The blood pump according to claim 13, wherein at least a first threshold temperature and a second, higher threshold temperature are established in the electronics, and a first amount of down-regulation and/or blood flow rate increase is provided if the first threshold temperature is surpassed, and a second, larger amount of down-regulation and/or blood flow rate increase is provided if the second threshold temperature is surpassed.

15. The blood pump according to claim 1, wherein the electronics further comprise multiple electronic subassemblies distributed to avoid relatively higher heat generating subassemblies being next to each other.

16. The blood pump according to claim 1, further comprising fins thermally coupled to the electronics.

17. The blood pump according to claim 1, further comprising a pump housing and wherein the electronics further comprise a module is at least partially positioned about the pump housing, and a major axis of the module is oriented relatively orthogonal to blood flow through the housing to thereby minimize the amount of heat received by each blood cell passing through the housing.

18. The blood pump according to claim 17, wherein the housing includes a thinned wall portion at heat transfer locations.

19. The blood pump according to claim 17, wherein the housing includes a minimum wall thickness at heat transfer locations for preventing undesired temperature spikes.

20. A method for data transfer in a blood pump system comprising:
integrating a sensor controller into a blood pump; and
modulating data of the sensor controller onto the motor current.
